(19) United States
(12) Patent Application Publication
(10) Pub. No.: US 2006/0142737 A1
(43) Pub. Date: Jun. 29, 2006

(54) Catheter

(76) Inventor: Allan Tanghoj, Kokkedal (DK)

Correspondence Address: JACOBSON HOLMAN PLLC, 400 SEVENTH STREET N.W., SUITE 600, WASHINGTON, DC, 20004, US

(21) Appl. No.: 10/537,014

(22) PCT Filed: Nov. 28, 2003
(86) PCT No.: PCT/DK03/00820

§ 371 Date: Mar. 6, 2006

(30) Foreign Application Priority Data
Nov. 29, 2002 (DK) PA 2002 01850

(51) Int. Cl. A61M 27/00; A61F 5/44

(52) U.S. Cl. 604/544; 604/329

(57) ABSTRACT

The invention provides a catheter, e.g. a urinary catheter, comprising two parts joined in a fixed connection. One part is made for manipulation of the other part, which other part is made for insertion into the body. The catheter comprises a sleeve attached to the outer surface of one of parts and which protects against bacterial contamination of the catheter and against smudging of surroundings with lubricants, water or similar friction reduction substances which may be pre-applied to the catheter to facilitate safe insertion. The invention fuirther provides a sleeve for the above mentioned catheter.

### INTRODUCTION

The present invention relates a catheter for urinary catheterisation, e.g. for catheterization of a person. In particular, the invention relates to a catheter with a conduit for draining body fluid from a proximal, insertable, end of the catheter to an opposite distal end of the catheter. The catheter comprises first and second parts wherein the first part forms the proximal end, and the second part forms the distal end, the first and second parts having different cross-sectional shape to support different use, i.e. for insertion into a urinary tract and for handling, respectively. The parts are joined in a fixed connection.

### BACKGROUND OF THE INVENTION

A urinary catheter normally forms an oblong catheter tube or a conduit with a similar function for insertion into a urinary canal of an individual. Some catheters are formed with a relatively short conical connector part e.g. for connecting the catheter to elongate tubes or drainage containers for collecting urine. By gripping the oblong catheter tube, the catheter is manipulated and inserted into the urinary canal.

Existing catheters are designed to give substantially no sensation of pain during insertion. Accordingly, catheters are typically provided with a smooth and slippery surface which is optimised for safe and comfortable insertion into the urethra. However, due to the slipperiness of the surface it can be difficult, not least for a user with reduced dexterity, to manipulate the catheter to avoid contamination of the catheter during use. Therefore it may happen that the catheter gets contaminated during unpacking thereof.

Sometimes, catheters are made with a handle portion to manipulate an insertable part of the catheter. The shape of the handle portion normally facilitates a more firm grip of the catheter. U.S. Pat. No. 5,653,700 discloses a catheter of this kind. Often, however, a friction reducing substance, e.g. gel or water, which is applied to the insertable part of the catheter, contaminates the handle portion and makes it slippery and difficult to use for people having a reduced dexterity.

In U.S. Pat. No. 5,226,530, the above-mentioned problem has been reduced by a package with a compartment for separation of the lubricious substance from the handle part of the catheter. This attempt to reduce contamination of the handle, however, does not solve the problem of unpacking the catheter, and in some packages, the unpacking procedure gets even more difficult with a following increased risk of contaminating the insertable tip part of the catheter.

In other products the contamination of the handle portion has been solved by providing a handle formed separately from the catheter for attachment to the catheter prior to the insertion. Unfortunately, handles which are separate from the catheter imply other problems both with respect to the manufacturing costs and with respect to handling of two separate components when attaching the handle to the catheter. In addition, separation of the catheter into two sepnarate components implies an increased risk of contamination, in particular, if the handle part is reused. Furthermore, division of the catheter into a handle part separate from an insertable part does not solve the problems of unpacking.

### DESCRIPTION OF THE INVENTION

It is an object of a preferred embodiment of the invention to overcome the aforementioned disadvantage of the known catheters, and to provide a catheter which is simple to unpack.

Accordingly, the present invention in a first aspect provides a catheter of the kind mentioned in the introduction wherein the insertable part is at least partly encapsulated in a sleeve which is attached, e.g. to the outer surface of the catheter leaving at least a portion of the second part uncovered by the sleeve. The invention could be applied e.g. to a urinary catheter.

Since the sleeve is attached to the catheter, the user may conveniently bring along and handle the catheter via direct contact with the second part, i.e. the handle part of the catheter. During such handling, the attached sleeve protects the insertable first part of the catheter from contamination. Attachment of the sleeve to the catheter reduces the risk that the sleeve accidentally falls off when handling the catheter prior to use.

Since the second part, i.e. the handle part, is fixed to the first part, the second part can be used actively during the unpacking of the catheter by holding the second part in a firm grip while removing the sleeve from the insertable part. In other words, the second part supports not only the insertion of the catheter into the urinary tract but also supports safe unpacking of the catheter.

Preferably, the length of the second part is at least ⅓ of the total length of the catheter, and preferably, the second part is not covered by the sleeve when the sleeve is attached to the catheter. The total length of the catheter, i.e. of the sum of the length of both parts of the catheter is preferably at most 160 mm. The length of the first part could in fact be reduced to the minimum length required to open the sphincter of the urinary tract of the user. Accordingly, the first part could be between 40 and 100 mm, e.g. between 50 and 80 mm. such as 70 mm. The length of the second part should allow a firm grip. It has been found that a gripping part with a length of 50 mm. or more allows the user to hold and to manipulate the catheter. Therefore, the length of the second part could e.g. be between 50 mm. and 110 mm. The catheter is thus substantially shorter than existing catheters.

The first and second parts may have a mutual longitudinal axis, or the second part may extend at an angle to the first part. The first and second parts can be designed differently, i.e. the size and/or the shape of the two parts can be different. As an example, the first part as well as the second part may have a circular cross-sectional shape, and a larger radial size of the second part could make handling of this part easier. Alternatively, the first part may have a circular shape while the second part has a non-circular shape, and vice versa.

The first and second parts are fixed to prevent separation of the parts. This makes the manufacturing, the packaging, and the use of the catheter easier. The parts may be joined by gluing or they could be produced as one single component, e.g. moulded in a pressure moulding process. The parts may be made from materials with different characteristics towards softness and/or rigidity. The insertable part may be made from a soft polymeric material allowing easy insertion into the tortuous urinary tract of male users or it may be made from a rigid polymeric material allowing easier insertion into the almost straight urinary tract of female users. The insertable part may further have a low frictional surface e.g. by provided by a low frictional material, or provided by a coating covering the insertable part, e.g. a hydrophilic coating, or a coating of gel, or similar lubricious substances. The handle part may, on the contrary, be made from a low frictional material, or it may have a surface coating preventing sliding between the fingers of the user, or it may have a surface pattern facilitating a better grip. The handle part could be made from a material which is rigid compared to the insertable part of the catheter.

The cross-sectional area of the second part may preferably be larger than the cross-sectional area of the first part, e.g. such that the ratio between the cross-sectional areas of the two parts is in the order of 1:10 or more, i.e. so that the second part has a cross-sectional area which is up to 10 times, or more, larger than the cross-sectional area of the first part. Alternatively, or furthermore, the second part could be made from a material which is different from the material of the first part, e.g. a material which supports firm gripping, e.g. a material which exhibits a relatively large frictional resistance against sliding between the fingers of the user, e.g. a soft resilient rubber material. In order further to improve the grip, the second part may be provided with knobs, grooves, slots or similar grip improving surface shapes or structures. The second part may coextend the first part to form a catheter with a substantially oblong linear shape, or the second part may form an angle to the first part to form a catheter with a non-linear shape.

The first part of the catheter is to be inserted into a urinary canal, and therefore this part may be made in a flexible material so that, during insertion, it follows the course of the canal without posing substantial pain or malaise. To ensure easy insertion of this first part, the second part could be less flexible, i.e. have a higher bending moment than the first part. Also to ensure easy insertion, the second part could have an ergonomically improved shape, e.g. comprising one or more depressions helping to lock the second part between the fingers of the user.

The first and/or the second part of the catheter and/or the sleeve, or at least a part of one of the parts could be made from a thermoplastic elastomer or other thermoplastic materials, or from a curable elastomer material, or from any mixture or combination thereof. Thermoplastic elastomer materials may comprise materials like Polyurethane elastomers (e.g. Estane™), Polyetherblockamide elastomers (e.g. Pebax™), Polyester elastomers (e.g. Hytrel™), Polyolefin elastomers (e.g. Santoprene™ and e.g. Engage™), Polystyrene elastomers (e.g. Kraton™ compounds) and PP elastomers with controlled tactic and atactic domains. Other thermoplastic materials may comprise PVC, e.g. plasticized PVC, Polyethylene homo- or co-polymers, polypropylene homo- or co-polymers, Polyamide types, Polyester types, fluorine-containing thermoplastic materials such as fluorine-containing elastomers among others. Curable elastomer material may comprise silicone elastomers and curable polyurethane elastomers among others.

In order to maintain sterility of the first part even during handling of the catheter via direct contact with the second part, the sleeve may be attached to the outer surface of the catheter in a seal, e.g. forming a liquid and/or bacteria tight encapsulation of the first part or at least of an insertable part thereof. For this purpose, it may be required to seal also the openings which are typically provided in the tip of the insertable part for draining fluid from the body and into the catheter conduit. The openings in the proximal, insertable, end of the catheter may be sealed via a cap covering the tip part and the openings therein. Such a cap could be integrated into the sleeve. In an alternative embodiment, the conduit is sealed in the opposite, distal, end of the catheter, e.g. via a sheet of a foil which is fastened to the end flange of the catheter, e.g. in a way allowing the foil to be pealed off from the flange.

The sleeve could be dimensionally stable, i.e., due to the stiffness of the sleeve material, the shape thereof is maintained during handling. As an example, the sleeve may have a rigid bottom part with a stiff sidewall extending there from. The sidewall could have a cylindrical shape, and it could be circular or non-circular in a cross-sectional view. The sleeve, or at least the sidewall thereof, could be made with a rigidity preventing the sidewall to be squeezed into engagement with the outer surface of the insertable part of the catheter. The sleeve could have an integrated clip fastened to the sleeve and functioning therewith in a manner similar to the clip of a cap known for a ballpen. The sleeve may also have other fastening means, e.g. a hook or a lug for hanging the catheter onto a corresponding hook, e.g. of a wall of a restroom. In use, the catheter is attached e.g. to a hook on the wall and by pulling the second part, i.e. the handle part, the catheter is released from the sleeve part which remains hanging in its fastening means on the hook.

The sleeve may be attached to the catheter e.g. by:
mechanically interlocking parts, e.g. a protrusion of one part engaging a groove of the other part, or by means of screw threads for screwing the parts to and from each other, or
fixed attachment wherein the parts are connected e.g. by glue with a strength which allows the user to pull off the sleeve, or wherein the parts are moulded together, e.g. wherein the sleeve is moulded directly onto the catheter.

For supporting removal of the sleeve from the catheter, at least one of the catheter and sleeve may have a non-circular cross-sectional shape. Such a non-circular shape may support a better grip e.g. for twisting the parts apart.

It may sometimes be necessary to collect the urine drained from the bladder. This may be achieved either by including a connection means in the catheter, e.g. as a part of the second part of the catheter such that it can be connected to a reservoir or a receptacle. Alternatively, a fixed fluid connection between the outlet opening of the catheter and a bag for collection of the body fluid may be established, e.g. by moulding a bag and the catheter in one piece, or by connecting the catheter and a bag fixedly via a tubular conduit or any other type of conduit.

In order to ease the insertion, at least the first part of the catheter or at least a part of the first part of the catheter may have a surface with low frictional characteristics. On this part, the surface could correspond to the surface of a regular gel-lubricated catheter, a hydrophilic catheter, or any catheter known per se. However, if the entire surface of the catheter is slippery, it may be difficult, especially for persons with reduced dexterity, to manipulate the catheter. Therefore, the second part of the catheter is preferably not provided with a surface with a low frictional characteristic but, as described previously, rather with a highly frictional surface, e.g. made from a soft resilient rubber material.

The catheter or at least the first part thereof may be wrapped in a packing so that the catheter is sterile prior to breaking the packaging seal, i.e. prior to insertion. The sleeve may form an integrated part of such a package in the sense that the package contains two or more compartments, i.e. a first compartment enveloping the first part of the catheter, and a second compartment enveloping the second part of the catheter. The two compartments may be sealed from each other to prevent a lubricating or sterilising substance in the first compartment to contaminate the second part of the catheter, i.e. the handling part. Preferably, the first part of the catheter is packed in a gas impermeable material preventing a lubricating substance applied to the first part to dry out, whereas the second part of the catheter is packed in a material which is at least partly transparent, thus allowing the user to see the second part through the material.

According to a second aspect, the invention provides a sleeve for a catheter with inlet openings in its insertable end, said sleeve forming an oblong body with a cavity for the catheter and an opening for inserting the catheter into the cavity, characterised in that the cavity forms a cap portion covering the inlet openings of the catheter when the catheter is arranged in the cavity. The sleeve could be made with any of the fastening means mentioned for the first aspect of the invention.

### DETAILED DESCRIPTION OF THE INVENTION

A preferred embodiment of the invention will now be described in details with reference to the drawing in which:

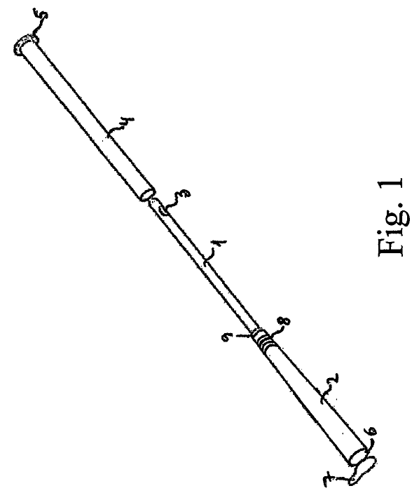

FIG. 1 shows a catheter according to the present invention,

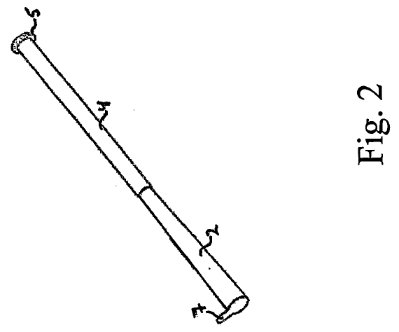

FIG. 2 shows the catheter of FIG. 1 wherein the sleeve is attached to the catheter,

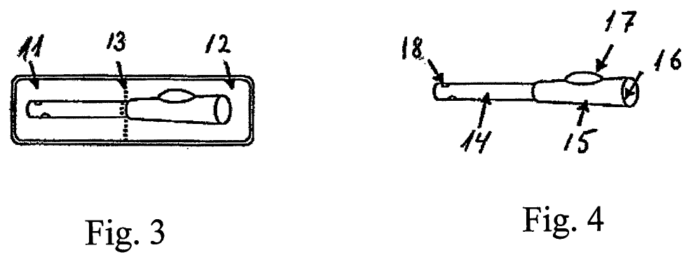

FIG. 3 shows a catheter wrapped in a two-compartment packaging,

FIG. 4 shows an alternative embodiment of the first and second parts of the catheter,

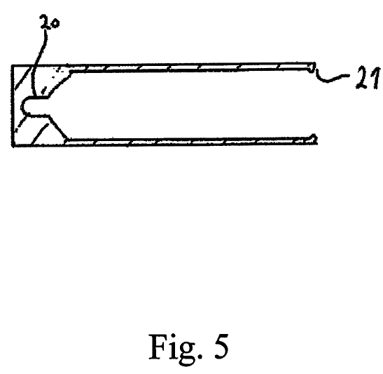

FIG. 5 shows an embodiment of the sleeve including a cap for covering openings of the catheter, and

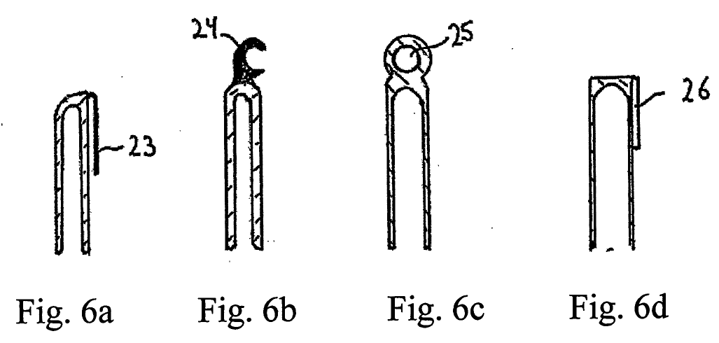

FIG. 6 shows four different embodiments of fastening means for attaching the sleeve to fittings or fixtures, e.g. of a restroom.

The catheter in FIG. 1 is for draining a body fluid, namely a urinary catheter for draining urine from a natural or artificial urinary tract. The catheter has a first part 1 forming the proximal, insertable end of the catheter, and a second part 2 forming a handle part of the catheter. The first and second parts have different shape corresponding to their intended use. The first part is oblong and has an inlet opening 3 for draining urine from the bladder into an internal conduit extending through both part of the catheter, and the first part is slim when compared to the second part. The first part is covered by a sleeve 4 which is detachably attached to the outer surface of the catheter (in FIG. 1, the sleeve is removed and the catheter is ready for insertion into the urinary tract). The disclosed sleeve is cylindrical, and has an outward flange 5 supporting removal of the sleeve from the catheter. The internal conduit connects the inlet opening with the outlet opening 6 opposite the inlet opening in the second part. The outlet opening is covered by a foil 7 which is attached in a manner which allows pealing. A ribbed portion 8 gives the user a tactile indication of the transition between the first and the second part. The first and second parts are joined in a joint 9, e.g. by gluing or welding or the parts are made in one piece.

FIG. 2 shows the catheter of FIG. 1, wherein the sleeve is attached to the catheter. The second part 2 is not covered by the sleeve. The sleeve fastens to the second part via an inwardly extending flange (not shown) engaging the ribbed portion 8.

FIG. 3 shows a catheter wrapped in a two-compartment package. A first compartment 11 is separated from a second compartment 12 by a leak tight seal 13. The package is composed of two foils joint along an edge in a seal allowing the foils to be pealed apart. The edge joint around the second compartment may be weak compared to the edge joint around the first compartment. This allows the user to easily open the second compartment to get access to the handle part. When access to the handle has been established, the user may get a firm hold in the handle and thus be able to open the more stronger adhering first compartment seal.

The catheter of FIG. 4 comprises first and second parts 14, 15. The second part, which forms the handle of the catheter, is tapered outwardly towards the distal, non-insertable, end 16, and a knob 17 support a better grip around the second part. The openings 18 are located in the proximal end of the catheter for draining liquid from the body into the catheter.

FIG. 5 shows a cross sectional view of one embodiment of the sleeve comprising a cap portion 20 for covering the openings of the proximal, insertable, end of the catheter. The radially inward flange 21 serves to catch a groove or protrusion of the catheter to lock the sleeve thereto.

FIGS. 6a-6d show four different embodiments of fastening means for attaching the sleeve to fittings or fixtures, e.g. of a restroom, for supporting removal of the sleeve from the catheter. FIG. 6a shows, a clip similar to the clips 23 known from a ballpen can be used for attaching the sleeve to an edge, a hook, a nail, or any similar fitting or fixture of a room. FIG. 6b shows a sleeve with a hook 24. FIG. 6c shows a sleeve with a lug 25, and FIG. 6d shows a sleeve with a piece of a fastening tape 26, e.g. an adhesive tape or Velcro™ tape.

1. A catheter with a conduit for draining body fluid from at least one inlet opening in the proximal, insertable, end of the catheter to at least one outlet opening, said catheter comprising first and second parts, wherein the first part forms the proximal end, and the second part forms the distal end, the first and second parts having different cross-sectional size and/or shape and being joined in a fixed connection, and the first part is at least partly encapsulated in a sleeve to leave at least a portion of the second part of the catheter uncovered by the sleeve, characterized in that the sleeve is attached to the catheter reducing the risk that the sleeve accidentally falls off when handling the catheter prior to use.

2. A catheter according to claim 1, wherein the sleeve is detachably attached to the outer surface of the catheter.

3. A catheter according to claim 1, wherein the sleeve forms at least a part of a liquid tight encapsulation of the first part.

4. A catheter according to claim 1, wherein the first part is entirely encapsulated by the sleeve.

5. A catheter according to claim 1, wherein the at least one inlet opening is sealed by a detachable closure.

6. A catheter according to claim 5, wherein the closure forms part of the sleeve.

7. A catheter according to claim 4, wherein the at least one outlet opening is sealed by a detachable closure.

8. A catheter according to claim 1, wherein the first and second parts are made from different materials.

9. A catheter according to claim 1, wherein the length of the second part constitutes at least ⅓ of the total length of the catheter.

10. A catheter according to claim 1, wherein the first and second parts are made in one piece.

11. A catheter according to claim 1, wherein the second part is made with a higher bending moment than the first part.

12. A catheter according to claim 1, wherein the second part is made with a higher surface friction than the first part.

13. A catheter according to claim 1, wherein the second part in a radial direction extends 2-10 times the radial size of the first part.

14. A catheter according to claim 1, wherein the second part comprises connection means for connecting collecting means for collection of the body fluid.

15. A catheter according to claim 1, of claims 1-13, wherein the outlet opening is in fluid connection with a receptacle for collection of the body fluid.

16. A catheter according to claim 1, wherein the sleeve is made from a dimensionally stable material.

17. A catheter according to claim 1, wherein the sleeve forms part of a package for encapsulation of the first and the second parts in individual compartments.

18. A catheter according to claim 1, wherein the sleeve or the first or the second part is made from a thermoplastic material.

19. A catheter according to claim 1 ant wherein sterility is maintainable within the sleeve during direct contact with the second part.

20. A catheter according to claim 1, of the preceding claims, made for draining urine from a bladder or urinary tract.

21. A sleeve for a catheter with inlet openings in its insertable end, said sleeve forming an oblong body with a cavity for the catheter and an opening for inserting the catheter into the cavity, characterised in that the cavity forms a cap portion covering the inlet openings of the catheter when the catheter is arranged in the cavity.
